(19) United States
(12) Patent Application Publication
(10) Pub. No.: US 2012/0195862 A1
(43) Pub. Date: Aug. 2, 2012

(54) BIOMECHANICAL INDUCTION OF HEMATOPOIESIS

(75) Inventors: George Daley, Weston, MA (US); Olaia Naveiras, Lausanne (CH); Guillermo Garcia-Cardena, Cambridge, MA (US); Luigi Adamo, St. Louis, MO (US)

(73) Assignees: CHILDREN'S MEDICAL CENTER CORPORATION, Boston, MA (US); BRIGHAM AND WOMEN'S HOSPITAL, INC., Boston, MA (US)

(21) Appl. No.: 13/264,898

(22) PCT Filed: Apr. 16, 2010
(86) PCT No.: PCT/US2010/031382

§ 371 Date: Mar. 28, 2012

Related U.S. Application Data
(60) Provisional application No. 61/170,480, filed on Apr. 17, 2009.
(60) Provisional application No. 61/172,492, filed on Apr. 24, 2009.
(60) Provisional application No. 61/178,247, filed on May 14, 2009.

(51) Int. Cl. C12N 5/0789; A61K 35/14

(52) U.S. Cl. 424/93.7; 435/377

(57) ABSTRACT

The present invention provides methods and compositions for increasing the hematopoietic potential of a population of hematopoietic progenitor cells, vascular cells, and or hemogenic endothelium, by exposing the cells to at least one external biomechanical stimulus. More specifically, the application of shear stress to hematopoietic progenitor cells or endothelial cells stimulates hematopoiesis, with or without concurrent application of other extrinsic modulators of hematopoiesis.

### CROSS REFERENCE TO RELATED APPLICATIONS

This application claims the benefit of U.S. Provisional Application No. 61/170,480, filed Apr. 17, 2009; and 61/178,247, filed May 14, 2009; and U.S. Provisional Application No. 61/172,492, filed Apr. 24, 2009, each incorporated herein by reference.

### GOVERNMENT SUPPORT

This invention was made with governmental support under grant No. HL-076686, awarded by the National Institutes of Health. The U.S. government has certain rights in the invention.

### FIELD OF THE INVENTION

The present invention provides for methods and compositions that increase hematopoietic cell populations. More specifically, exposing hematopoietic stem cells, hemogenic endothelial cells, or any of their developmental cell precursors to biomechanical force induces the expansion and/or differentiation of the hematopoietic cell population.

### BACKGROUND

Stem cell research holds extraordinary potential for the development of therapies that may change the future for those suffering from diseases such as leukemia, diabetes, and anemia. Much research focuses on the exploration of stem cell biology as a key to treatments for diseases. Through an understanding of the role of stem cells in normal development, researchers seek to capture and direct the innate capabilities of stem cells to treat many conditions. Research is on-going in a number of areas simultaneously: examining the genetic and molecular triggers that drive embryonic stem cells to develop in various tissues; learning how to push those cells to divide and form specialized tissues; culturing embryonic stem cells and developing new lines to work with; searching for ways to eliminate or control Graft vs. Host Disease by eliminating the need for donors; and generating a line of universally transplantable cells.

Hematopoietic progenitor cells are derived during embryogenesis in distinct regions where specific inductive events convert mesoderm to blood stem cells and progenitors. Following initiation of the heartbeat in vertebrates, cells lining the inner aspect of the dorsal aorta, the placental vessels, and the umbilical and vitelline arteries, begin to manifest hematopoietic properties. It remains unknown, however, if the biomechanical forces imposed on the vascular wall at this developmental stage act as a determinant of hematopoietic potential. Thus, there remains a need to elucidate the relationships between particular biomolecules, chemical agents, and biomechanical forces that show promise in manipulating hematopoietic progenitor cells for a desired purpose, such as increasing a hematopoietic progenitor cell population in vitro and/or ex vivo.

### SUMMARY

The present invention harnesses the critical role of biomechanical forces in hematopoietic development. More specifically, the present invention provides for methods of stimulating hematopoietic stem cell differentiation and growth by applying biomechanical forces to a hematopoietic progenitor cell population or to a cell population able to differentiate along the hematopoietic lineage. The embodiments of the present invention provide methods for increasing the hematopoietic potential of progenitor cells in vitro or ex vivo, comprising stimulating a cell population comprising hematopoietic progenitor cells or cells endowed with the ability to differentiate into hematopoietic progenitors with biomechanical forces, such as circumferential stress, hydrodynamic pressure, and shear stress. In particular embodiments, a hematopoietic progenitor cell population is subjected to wall shear stress.

A particular embodiment of the invention provides a method for producing hematopoietic cells comprising obtaining a culture of embryonic hematopoietic progenitor cells or cells committed along vascular lineage, and stimulating the cells in an in vitro system that subjects the cells to wall shear stress, and culturing the cells, thereby producing hematopoietic cells. For example, the magnitude of the shear stress applied to the cells may range from 0 dyne/cm² to 150 dyne/cm², inclusive, such as 0 dyne/cm² to 35 dyne/cm², 0 dyne/cm² to 10 dyne/cm², or 0 dyne/cm² to 5 dyne/cm², inclusive. Cells may be stimulated for at least 1 hour to 72 hours, inclusive, such as, for example, from 1 hour to 48 hours, inclusive.

The methods of the invention can be used with a variety of animal or human cell populations containing hematopoietic stem cells, hematopoietic progenitors or vascular progenitors including embryonic stem cells, umbilical cord blood stem cells, unrestricted somatic stem cells derived from human umbilical cord blood, induced stem cells, somatic stem cells, mesenchymal stem cells, mesenchymal progenitor cells, hematopoietic stem cells, hematopoietic lineage progenitor cells, endothelial stem cells, placental fetal stem cells, hemogenic endothelial cells, and endothelial progenitor cells.

In some embodiments, the hematopoietic progenitor cell population may be obtained from embryoid bodies, peripheral blood, cord blood, bone marrow, amniotic fluid, chorionic villus tissue, placenta, or other hematopoietic stem cell niches. In particular embodiments, hematopoietic stem cells express CD34, CD41, c-kit, Flk1, PECAM, and/or MECA32 markers.

The invention also provides methods of treating a subject in need of improved hematopoietic capability, by stimulating a cell population comprising hematopoietic progenitor cells with biomechanical forces to induce hematopoiesis, thereby enhancing proliferation and/or hematopoietic differentiation of the progenitor cells; and administering the cells to the subject, thereby improving hematopoietic capability. In some embodiments, the biomechanical force is wall shear stress.

One embodiment of the invention provides for a method for producing, expanding and/or differentiating hematopoietic cells in a subject by obtaining a cell population enriched in hematopoietic precursors; exposing the cell population enriched in hematopoietic precursors to biomechanical stress to stimulate hematopoietic maturation, expansion and/or differentiation; and administering the stimulated population to the subject. The cells may also be contacted with a chemical modulator of hematopoiesis. Additionally, the subject may be a human.

Another embodiment of the invention provides for a method for producing long term repopulating hematopoietic stem cells in a subject, comprising differentiating stem cells; exposing cells derived from this differentiation process to shear stress to stimulate hematopoietic maturation or expansion; and administering the stimulated cell population to the subject. In aspects of this embodiment, the stem cells are induced pluripotent stem cells, autologous induced pluripotent stem cells, autologous multipotent stem cells, or allogeneic multipotent stem cells. Optionally, the cells may be contacted with a chemical modulator of hematopoiesis. In a particular aspect, the subject is a human.

Yet another embodiment provides for a method for producing cells of the hematopoietic lineage in a subject, comprising obtaining stem cells; differentiating the cells; exposing the cells derived from this differentiation to shear stress to stimulate hematopoietic maturation, expansion or differentiation of the cells; and administering the stimulated cell population to the subject. In aspects of this embodiment, the stem cells are induced pluripotent stem cells, autologous induced pluripotent stem cells, autologous multipotent stem cells, or allogeneic multipotent stem cells. The method may further include contacting the cells with a chemical hematopoiesis modulator. Additionally, the subject may be a human.

The invention also provides methods for producing hematopoietic stem cells or their differentiated cell lineages useful for screening agents for treatment of blood-borne diseases. For example, hematopoietic stem cells or their differentiated cell lineages are used to screen agents for treatment of malaria.

Optionally, the exposure to biomechanical force may be combined with exposure to at least one chemical hematopoiesis modulator. Thus, in some embodiments, at least one chemical modulator of hematopoiesis may be applied before, during, or after biomechanical stimulation. Example chemical modulators include Atenolol, Digoxin, Doxazosin, Doxycycline, Fendiline, Hydralazine, 13-hydroxyoctadecadienoic acid (13(s)-HODE), Lanatoside C, NG-monomethyl-L-arginine (L-NMMA), Metoprolol, Nerifolin, Nicardipine, Nifedipine, Nitric oxide (NO) or NO signaling pathway agonists, 1H-[1,2,4]oxadiazolo-[4,3-a]quinoxalin-1-one (ODQ), Peruvoside, Pindolol, Pronethalol, Synaptosomal protein (SNAP), Sodium Nitroprusside, Strophanthidin, Todralazine, 1,5-Pentamethylenetetrazole, Prostaglandin E 2 (PGE2), PGE2 methyl ester, PGE2 serinol amide, 11-deoxy-16,16-dimethyl PGE2, 15(R)-15-methyl PGE2, 15(S)-15-methyl PGE2, 6,16-dimethyl PGE2, 16,16-dimethyl PGE2 p-(p-acetamidobenzamido) phenyl ester, 16-phenyl tetranor PGE2, 19(R)-hydroxy PGE2, Prostaglandin B2, Prostacyclin (PGI2, epoprostenol), 4-Aminopyridine, 8-bromo-cAMP, 9-deoxy-9-methylene PGE2,9-deoxy-9-methylene-16,16-dimethyl PGE2, a PGE2 receptor agonist, Bapta-AM, Benfotiamine, Bicuclline, (2′Z,3′E)-6-Bromoindirubin-3′-oxime (BIO), Bradykinin, Butaprost, Cay10397, Chlorotrianisene, Chlorpropamide, Diazoxide, Eicosatrienoic Acid, Epoxyeicosatrienoic Acid, Flurandrenolide, Forskolin, Gaboxadol, Gallamine, Indanyloxyacetic acid 94 (IAA 94), Imipramine, Kynurenic Acid, L-Arginine, Linoleic Acid, LY171883, Mead Acid, Mebeverine, 12 Methoxydodecenoic acid, N-Formyl-Met-Leu-Phe, Prostaglandin E2 receptor EP2-selective agonist (ONO-AE1-259), Peruvoside, Pimozide, Pindolol, Sodium Nitroprusside, Sodium Vanadate, Strophanthidin, Sulprostone, Thiabendazole, Vesamicol, 1,2-Didecanoyl-glycerol (10:0), 11,12 Epoxyeicosatrienoic acid, 1-Hexadecyl-2-arachidonoyl-glycerol, 5-Hydroxydecanoate, 6-Formylindolo[3,2-B]carbazole, Anandamide (20:3,n-6), Carbacyclin, Carbamyl-Platelet-activating factor (C-PAF), or S-Farnesyl-L-cysteine methyl ester.

Other embodiments of the present invention provide for augmenting the hematopoietic colony-forming potential of embryonic stem cell-derived cells, using embryonic stem cells differentiated in vitro, by applying in vitro wall shear stress which increases the expression of CD31, Klf2 and Runx1 in CD41⁺/cKit⁺ blood progenitor cells. In other embodiments of the invention, shear stress increases the expression of hematopoietic markers in the aorta-gonads-mesonephros (AGM) of mouse embryos, in vitro and in vivo.

### DESCRIPTION OF THE DRAWINGS

FIG. 1 demonstrates that shear stress induces hematopoietic commitment from ES-derived cells. FIG. 1a, Experimental protocol used to induce hematopoietic differentiation from ES-derived cells in the presence of wall shear stress (WSS). ES cells are differentiated with the embryoid body method for 3.25 days, disaggregated and plated on gelatinized surfaces. Cell monolayers are exposed to shear stress and than collected on day 6 for further analysis. FIG. 1b, Quantitative TAQMAN® real-time PCR-based gene expression analysis in FACS-sorted CD41⁺/cKit⁺ EB-derived hematopoietic precursors. Exposure to WSS induces upregulation of the hematopoietic markers Runx1 (p=0.01), Myb (p=0.03), and Klf2 (p=0.001), n=3. FIG. 1c, Methylcellulose hematopoietic colony forming unit assay (CFU). WSS increases the frequency of hematopoietic progenitors in complete M3434 methylcellulose, n=4, p=0.01. Insert shows average distribution of hematopoietic colony types. CFU Granulocyte-erythroid-myeloid-megariocytes (CFU-GEMM), CFU Granulocytes/Macrophages/Granulocyte-Macrophages (CFU G/M/GM), CFU Erythroid (CFU-E). Bar graphs represent average +/−SEM. Color versions of some of these figures are available at Adamo et al., 459 Nature 1131-35 (2009).

FIG. 2 shows that nitric oxide production regulates the expansion of hematopoietic progenitors. FIG. 2a, Methylcellulose hematopoietic colony forming unit (CFU) assay. Pharmacological inhibition of nitric oxide (NO) synthesis with N(G)-nitro-L-arginine methyl ester (L-NAME) reduces by 50% the WSS-mediated increase in hematopoietic colony forming units, n=3, p=0.04. Insert shows the average distribution of colony types. FIG. 2b, L-NAME does not affect WSS-mediated Runx1 upregulation, n=3. FIG. 2c, in vivo colony forming unit (CFU) assay. Exposure of developing embryos to L-NAME from E8.5 to E10.5 leads to a reduction in hematopoietic progenitors in the aorta-gonad-mesonephros (AGM) region. 2 mM N(G)-nitro-D-arginine methyl ester (D-NAME) n=30, L-NAME n=32, p=0.0008. e.e., embryo equivalent. Insert shows average distribution of colonies per AGM. Bar graphs represent average +/−SEM.

FIG. 3 illustrates that AGM-derived hematopoietic progenitors respond to shear stress in vitro and in vivo. FIG. 3a, FACS analysis. WSS induces an increase in CD31⁺ cells in 2-dimensional primary AGM cultures. p=0.005, n=3, average +/−STDV. FIG. 3b, WSS induces upregulation of the hematopoietic markers Runx1 (p=0.01) and Klf2 (p=0.05) in FACS sorted AGM derived CD41⁺ hematopoietic progenitors. n=3 FIG. 3c, WSS modulates the differentiation of AGM derived hematopoietic progenitors as shown by an increase in absolute number of cells positive for the erythroid marker Ter119 (p=0.01) and for the lymphoid marker B220 (p=0.02), n=3. FIG. 3d, Shear stress induces a maturation of erythroid precursors as documented by cell morphology in cytospins, which show pycnotic erythroblast in WSS treated samples, and polychromatic erythroblasts in static cultures. FIG. 3e, AGMs isolated from E9.25 NCX1^{-/-} embryos show reduced gene expression levels of the specific hematopoietic markers Runx1 (p=0.02) and Klf2 (p=0.003) when compared to matched wild-type (Wt) or heterozygous (Het) littermate controls, n=13 NCX1^{-/-}, n=15.

FIG. 4a presents a diagram of a Dynamic Flow System (DFS) used to study the effect of shear stress on cultured hematopoietic progenitors. FIG. 4b, Shear stress pattern used to stimulate cultured cells. FIG. 4c, FACS analysis of cultured EB-derived cells demonstrating shear stress-induced increase in CD31⁺ cells. FIG. 4d, TAQMAN® gene expression assay of unfractionated EB-derived cells. WSS induces the hematopoietic markers Runx1 (p=0.01), Klf2 (p=0.01) and Sox17 (p=0.005), n=3. FIG. 4e, Shear stress does not activate the CDX4 transgene in the doxycycline iCDX4 cell line, n=3 FIG. 4d, Methylcellulose hematopoietic colony forming unit assay. D-NAME and L-NAME do not affect the number of CFUs in ES-derived cultures in static conditions, n=2 e, gene expression analysis. D-NAME and L-NAME do not affect Runx1 expression in static cultures of ES-derived cells, n=2.

FIG. 5a shows gene expression of unfractionated primary cultures of AGM derived cells. WSS induces the hematopoietic markers Runx1 (p=0.0005), Klf2 (p=0.007), Gata3 (p=0.0001) and Sox17 (0.028), n=3. FIG. 5b, Representative FACS plot from lineage analysis of the semi-adherent outflow from AGM-derived cells in primary culture (CD71 early erythroblasts, Ter119 late erythroblasts, Gr1 granulocytes, F4/80 macrophages, CD3 T-lymphocytes, B220 B-lymphocytes).

### DETAILED DESCRIPTION

It should be understood that this invention is not limited to the particular methodology, protocols, and reagents, etc., described herein and as such may vary. The terminology used herein is for the purpose of describing particular embodiments only. Unless otherwise defined herein, scientific and technical terms used in connection with the present application shall have the meanings that are commonly understood by those of ordinary skill in the art. Further, unless otherwise required by context, singular terms shall include pluralities and plural terms shall include the singular. Other than in the operating examples, or where otherwise indicated, all numbers expressing quantities of ingredients or reaction conditions used herein should be understood as modified in all instances by the term “about.”

All patents and other publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The embodiments of the present invention provide for a novel role for biomechanical forces as determinants of hematopoietic cell development, and provide for a new conceptual framework for modulating micro-environmental cues to sustain the specification or maturation of hematopoietic progenitors. Importantly, the compositions and methods of the present invention provide new approaches for the manipulation and production of hematopoietic progenitors in vitro, a key process for both implementing stem cell-based therapy for hematologic diseases and expanding the research platform for blood-borne diseases.

For example, expanding the number of hematopoietic stem cells is useful in transplantation and other therapies for hematologic and oncologic disease. As described in the methods herein, hematopoietic cell numbers are increased in vitro or ex vivo. A method of increasing hematopoietic cell numbers reduces the time and discomfort associated with bone marrow/peripheral stem cell harvesting and increases the pool of stem cell donors. Currently, approximately 25% of autologous donor transplants are prohibited for lack of sufficient stem cells. In addition, less than 25% of patients in need of allogeneic transplant can find a histocompatible donor. Umbilical cord blood banks currently exist and cover the broad racial make-up of the general population, but these banks are currently restricted to use in children due to inadequate stem cell numbers in the specimens for adult recipients. A method to increase hematopoietic progenitor cell numbers permits cord blood and other hematopoietic progenitor cell sources to be useful for subjects, including human adult patients, thereby expanding the use of allogeneic transplantation.

Hematopoietic stem cells, or hematopoietic progenitor cells, refer to immature blood cells having the capacity to self-renew and to differentiate into more mature blood cells comprising granulocytes (e.g., promyelocytes, neutrophils, eosinophils, basophils), erythrocytes (e.g., reticulocytes, erythrocytes), thrombocytes (e.g., megakaryoblasts, platelet producing megakaryocytes, platelets), and monocytes (e.g., monocytes, macrophages). Hematopoietic stem cells are interchangeably described as stem cells or progenitor cells herein. It is known that hematopoietic stem cells include pluripotent stem cells, multipotent stem cells (e.g., a lymphoid stem cell), and/or stem cells committed to specific hematopoietic lineages. The stem cells committed to specific hematopoietic lineages may be of T-cell lineage, B-cell lineage, dendritic cell lineage, Langerhans cell lineage and/or lymphoid tissue-specific macrophage cell lineage. In addition, hematopoietic stem cells also refer to long term (LT-HSC) and short term (ST-HSC) hematopoietic stem cells. A LT-HSC typically includes the long term (more than three months) contribution to multilineage engraftment after transplantation. A ST-HSC is typically anything that lasts shorter than three months, and/or that is not multilineage. Any of these HSCs can be used in any of the methods described herein.

Sources for hematopoietic stem cell expansion also include AGM, ESC and iPSC. ESC are well-known in the art, and may be obtained from commercial or academic sources (Thomson et al., 282 Sci. 1145-47 (1998)). iPSC are a type of pluripotent stem cell artificially derived from a non-pluripotent cell, typically an adult somatic cell, by inducing a “forced” expression of certain genes (Vogel & Holden, 23 Sci. 1224-25 (2007); Weber, 52 Pathol. Biol. 93-96 (2004)). ESC, AGM, and iPSC according to the present invention may be derived from animal or human sources. As discussed herein, the AGM stem cell is a cell that is born inside the aorta, and colonies the fetal liver. Signaling pathways can increase AGM stem cells make it likely that these pathways will increase HSC in ESC.

In the mouse, the first hemogenic areas appear in the yolk sac starting at day 7.5 of development (E7.5). Haar & Ackerman, 170 Anat. Rec. 199-223 (1971). The gene, runx1 (previously AML1 oncoprotein), is required for hematopoiesis, and between E9 and E10.5, additional Runx1⁺ hemogenic sites appear within specific sites of the developing vasculature. These events chronologically follow the establishment of circulation and the onset of vascular flow at day 8.5 (E8.5). Ji et al., 92 Circ. Res. 133-35 (2003). The developmental and anatomical relationships of hematopoietic precursors with the mechano-responsive vascular endothelium (Tavian et al., 1044 Ann. NY Acad. Sci. 41-50 (2005); Garin & Berk, 13 Endothel. 375-84 (2006)), and the temporal correlation between the establishment of circulation and the appearance and expansion of vascular hemogenic sites, support the present finding that biomechanical forces act as determinants of hematopoietic cell development.

For example, in some embodiments mouse embryonic stem cells (mES) were differentiated as embryoid bodies (EBs), a system that recapitulates the early stages of embryonic development. In EBs, cells first commit to mesoderm and express Flk1 (VEGF receptor 2), a tentative marker of hemangioblasts, and produce Flk1⁺ cells containing the earliest embryonic hematopoietic precursors. Kabrun et al., 124 Devel. 2039-48 (1997). Thus, EBs were cultured until the appearance of Flk1⁺ mesoderm on day 3.25 of differentiation, disaggregated, and plated on flat gelatinized surfaces. See Kabrun et al., 1997; Lengerke et al., 2 Cell Stem Cells 72-82 (2008). The type of biomechanical stimulation to apply on cultured cells was defined by focusing on the hemodynamic environment present in the aorta-gonad-mesonephros (AGM) region, a well-characterized hemogenic site at the onset of embryonic circulation. The pulsatile characteristics of flow in the aorta generate a complex interplay of distinct types of biomechanical forces including circumferential stress, hydrodynamic pressure, and shear stress.

Regarding these biomechanical forces, circumferential stress involves mechanical stress for rotationally-symmetric objects, resulting from forces acting circumferentially (perpendicular both to the axis and to the radius of the object). Circumferential stress can be calculated from a vessel's inside diameter, wall thickness, and chamber pressure, and can be manipulated by changing the internal or external pressure. See, e.g., Lee et al., 16 Arteriosclerosis, Thrombosis, & Vascular Bio. 1070 (1996). The vessel may be a cell, a cellular tissue, or a container holding a cell population. Hydrodynamic pressure describes the relationship between pressure and hydrostatic pressure (simply the weight of a fluid) in fixed-volume flow systems (liquids in motion). See, e.g., Podolak et al., 7 Innov. Food Sci. & Emerg. Techs. 28-31 (2006). Wall shear stress is a fluid shear stress created by the frictional force generated by viscous flow that acts on cells lining blood vessels (e.g., endothelial cells), and exerts profound effects on the structure and function of vascular endothelial cells. Garin & Berk, 13 Endothelium 375-84 (2006); Wang et al., 23 Nephrol. Dial. Transplant 2167-72 (2008).

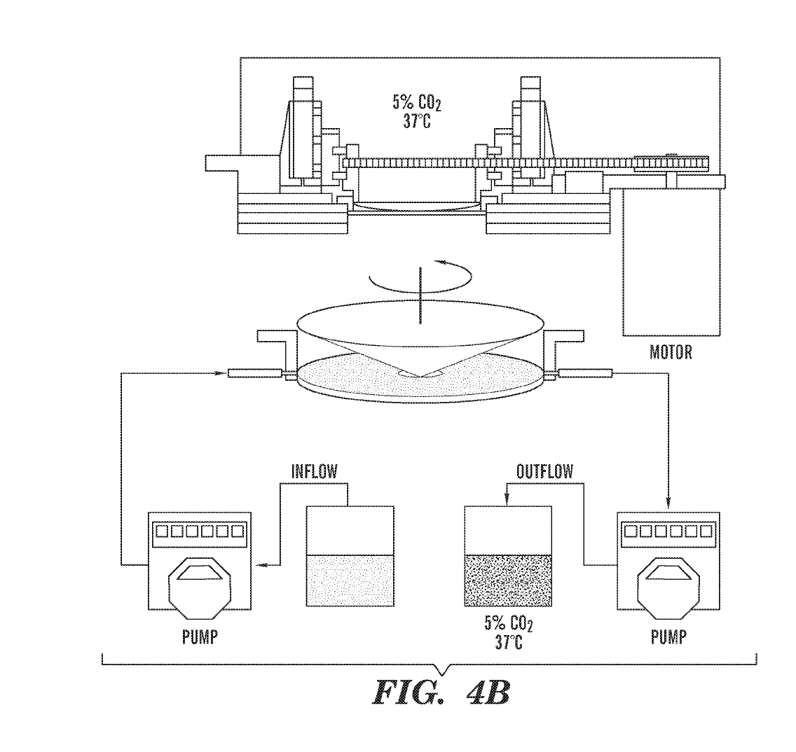

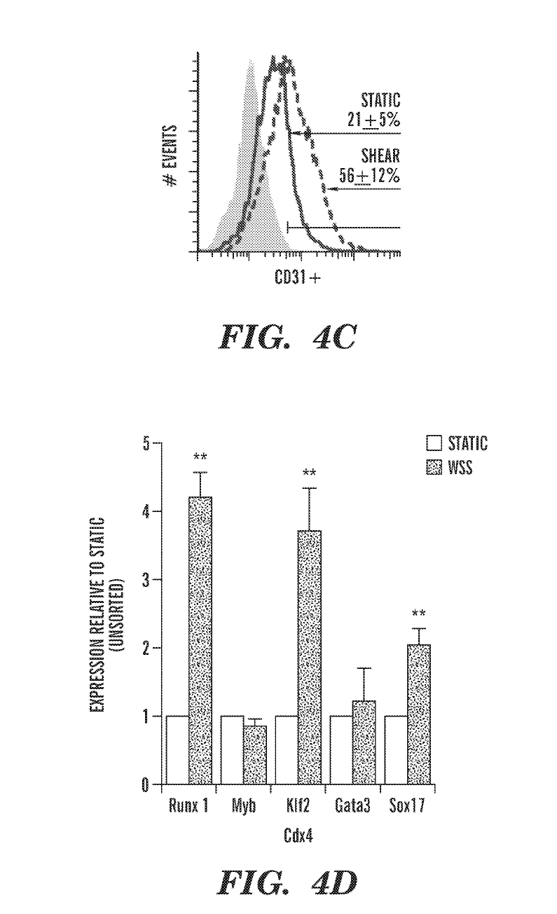

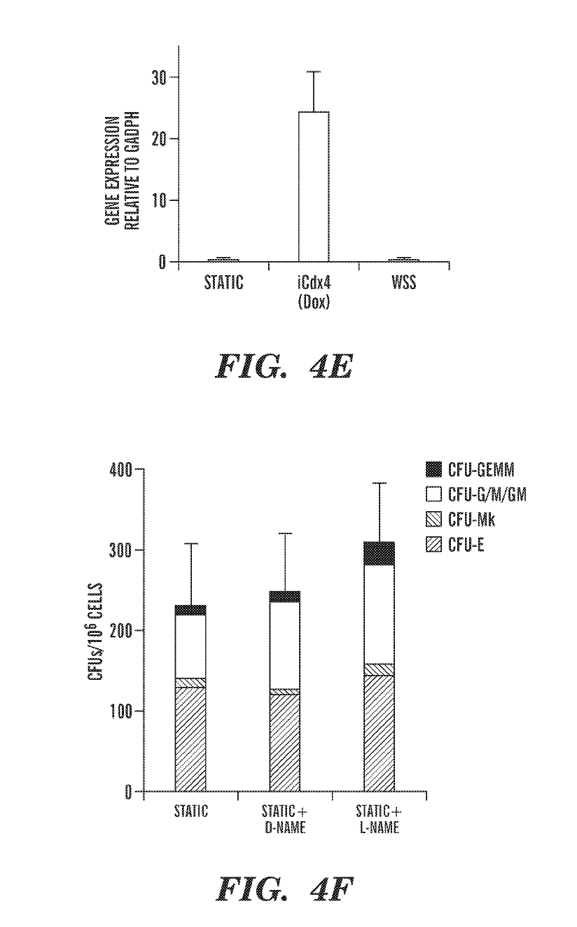

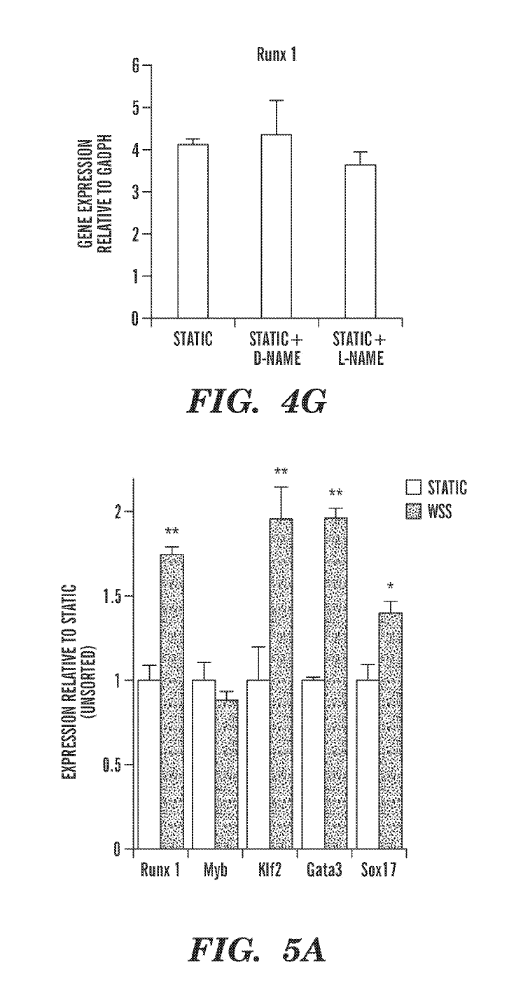

In some embodiments of the present invention, wall shear stress is used to enhance the hematopoietic potential of progenitor cells. Briefly, cultured cells were stimulated using a wall shear stress: a characteristic of the stress that acts along the dorsal aorta throughout the cardiac cycle at E10.5. The time-average of this value was estimated using previously published ultrasound biomicroscopy (UBM) data for the time average velocity over one cardiac cycle (velocity time integral, 4.74 mm, divided by the cycle length, 350 ms, and the earliest available values for dorsal aorta diameter, 0.33 mm). See Ji et al., 2003; Phoon et al., 283 Am. J. Physiol. Heart Circ. Physiol. H908-16 (2002). Calculating the circular Poiseuille flow solution for wall shear stress with these values yielded a dorsal aorta wall shear stress (WSS) at E10.5 of approximately 5 dyne/cm². (FIG. 4a). This approach assumed that the UBM velocity data represented center-line blood flow, and that Poiseuille flow assumptions are valid within the E10.5 dorsal aorta. Ku, 29 Ann. Rev. Fluid Mech. 399-434 (1997).

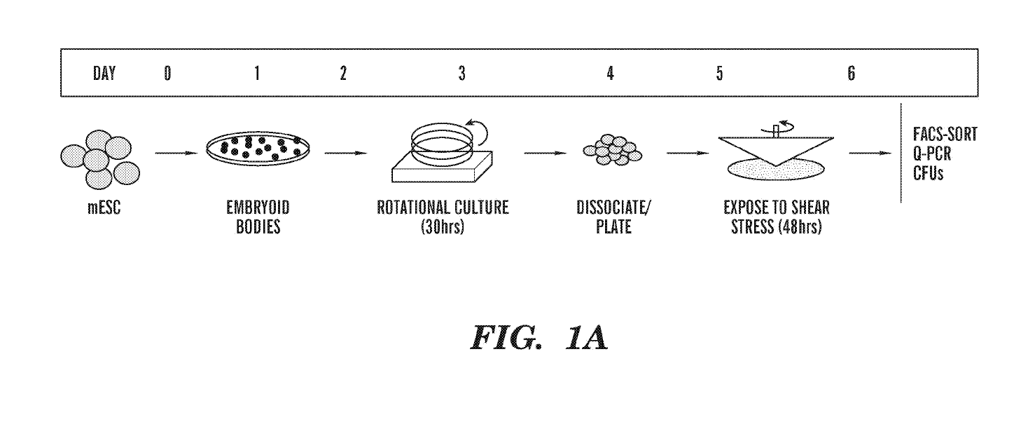

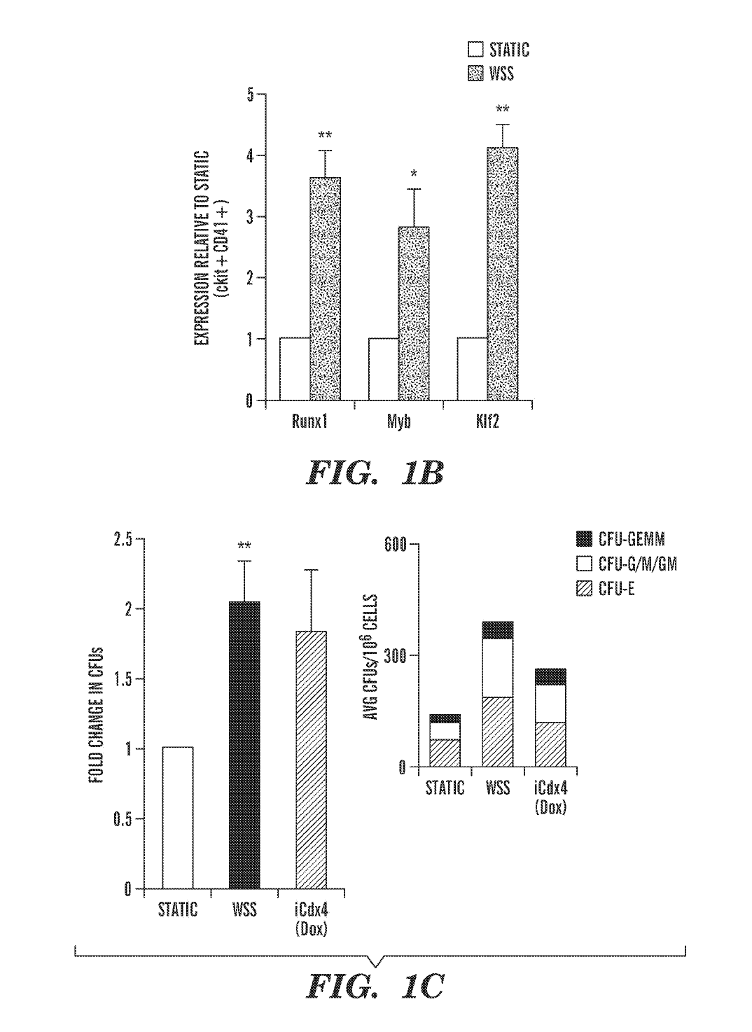

EB-derived cells were exposed to this biomechanical WSS stimulus or cultured under static (i.e., no shear stress) conditions for 48 hours (FIGS. 1a, 4b). Exposure of EB-derived cells to shear stress caused an increase in cells positive for CD31 (PECAM1), a marker of endothelial and hematopoietic lineages (FIG. 4c). See, Yamamoto et al., 288 Am. J. Physiol., Heart & Circ. Physiol. H1915-24 (2005). Additionally, CD41⁺/cKit⁺ hematopoietic precursors were FACS-sorted, because in the yolk sac and EBs, CD41 is coexpressed with embryonic HSC markers c-kit and CD34. Mikkola et al., 101 Blood 508-16 (2003). Gene expression analysis within this compartment demonstrated a strong shear stress-mediated upregulation of the transcription factors Runx1/Cbfa2 (4.6-fold) and Myb/Cbfb (2.8-fold), the prototypical markers of hemogenic sites (North et al., 16 Immunity 661-72 (2002)), and of Klf2 (4.1-fold), a gene previously shown to be both a driver of erythropoeisis (Basu et al., 106 Blood 2566-71 (2005)), and be mechano-activated in endothelial cells (FIG. 1b) (see also Parmar et al., 116 J. Clin. Invest. 49-58 (2006)).

Similar results were observed in the unsorted cell population (FIG. 4d). The functional significance of these gene expression changes were explored using hematopoietic colony forming assays. The iCdx4 ES cell line, which carries a doxycycline-inducible transgene that enhances differentiation to hematopoietic precursors, was used as a positive control. Wang et al., 102 P.N.A.S. 19081-86 (2005). Shear stress increased the frequency of hematopoietic colony-forming units (CFUs) when compared to static (no shear stress) conditions, with a magnitude comparable to the induction of Cdx4 with doxycycline (FIG. 1c). Interestingly, the Cdx4 transgene was not activated in cells exposed to shear stress (FIG. 4e). Similar results on gene expression and CFU frequency were obtained when Flk1⁺ cells enriched by magnetic cell-sorting were analyzed, indicating that the shear stress response occurs in the Flk1⁺ mesoderm. These observations demonstrate that biomechanical forces regulate the expression of markers of hematopoiesis in EB-derived hematopoietic precursors, and enhance embryonic stem cell-derived hematopoiesis.

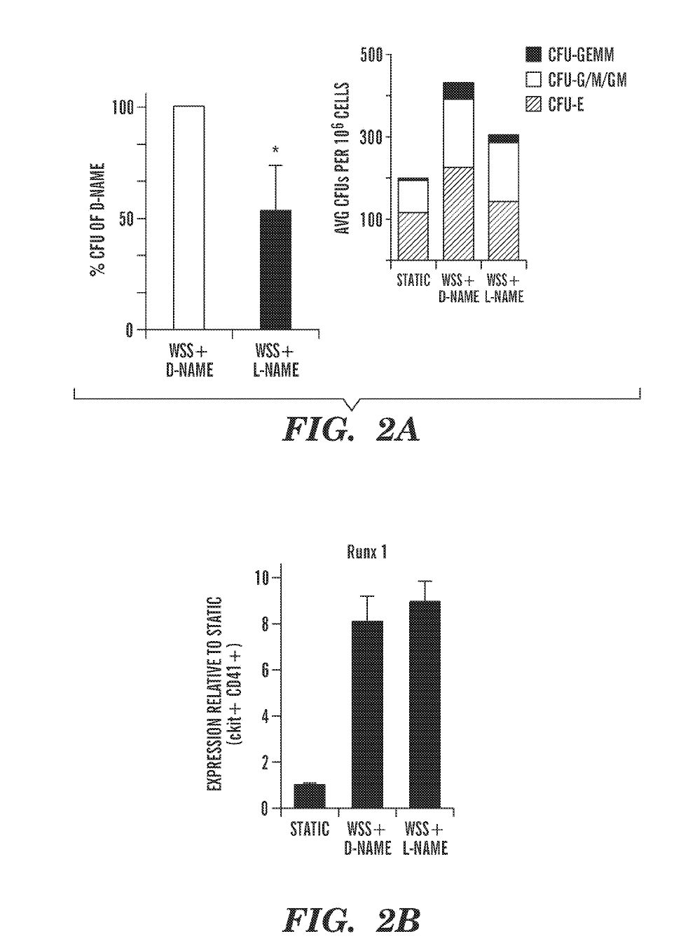

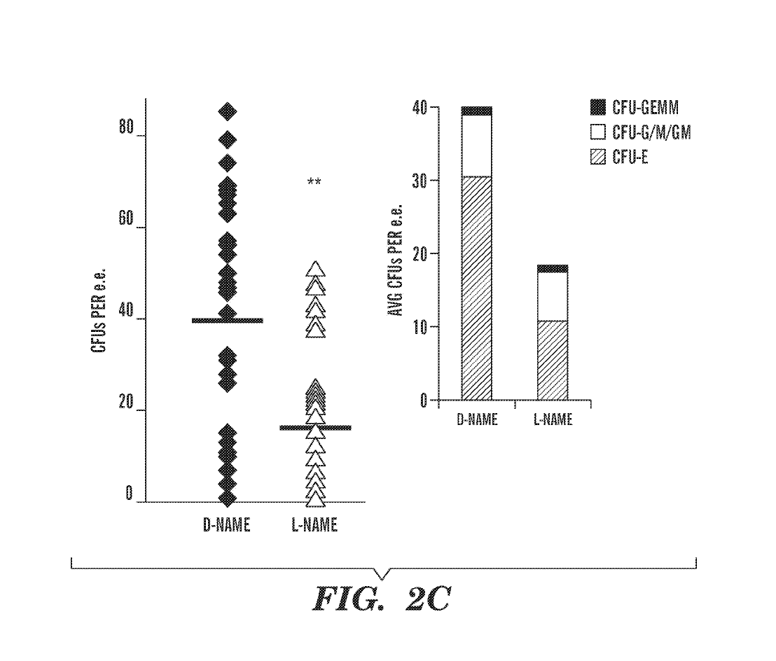

Mechanistic insight into this process was advanced by analyzing the role of nitric oxide (NO): a well-characterized signaling pathway strongly regulated by shear stress and a known modulator of hematopoiesis. Garcia-Cardena et al., 392 Nature 821-24 (1998); Shami & Weinberg, 87 Blood 977-82 (1996); WO 2009/155041. Inhibition of NO production via the nitric oxide synthase inhibitor, nitro-L-arginine methyl ester (L-NAME) (Rees et al., 86 P.N.A.S. 3375-78 (1989)), resulted in a significant reduction of the shear stress-induced enrichment in CFUs, as compared with cells treated with the inactive stereoisomer D-NAME (FIG. 2a).

Interestingly, L-NAME did not affect shear stress-induced Runx1 upregulation in CD41⁺/cKit⁺ cells, indicating that shear stress-mediated Runx1 upregulation is upstream of NO production (FIG. 2b). L-NAME and D-NAME had no effect on CFU formation or Runx1 expression in cells grown under static conditions (FIGS. 4f, 4g). The role of NO production on intraembryonic hematopoiesis was studied in vivo by administering L-NAME or D-NAME to pregnant mice for 48 hours, starting from the time of establishment of circulation (E8.5), then assessed the number of CFUs in the AGM regions of E10.5 embryos. As shown in FIG. 2c, systemic inhibition of NO production by L-NAME led to a marked decrease in the number of CFUs per AGM. This in vivo setting does not distinguish between the direct effect of NO inhibition on hematopoietic precursors and the alterations of embryonic hemodynamics that result from changes in vascular tone triggered by the systemic inhibition of nitric oxide synthases. Rees et al., 1989. Nevertheless, these data document that NO production is associated with the shear stress-mediated stimulation of hematopoietic progenitors in vitro, and also in vivo as seen by the numbers of hematopoietic precursors in the AGM region.

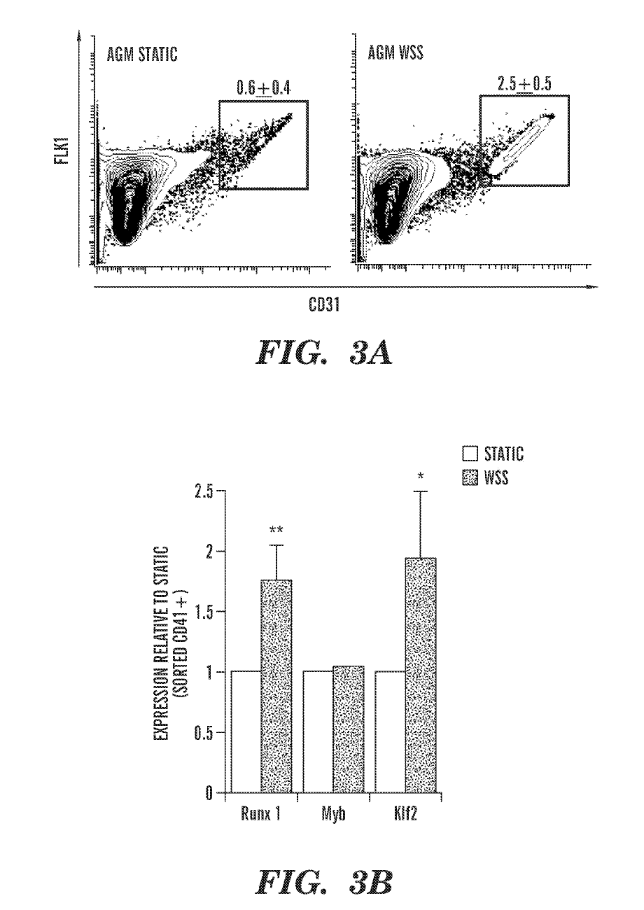

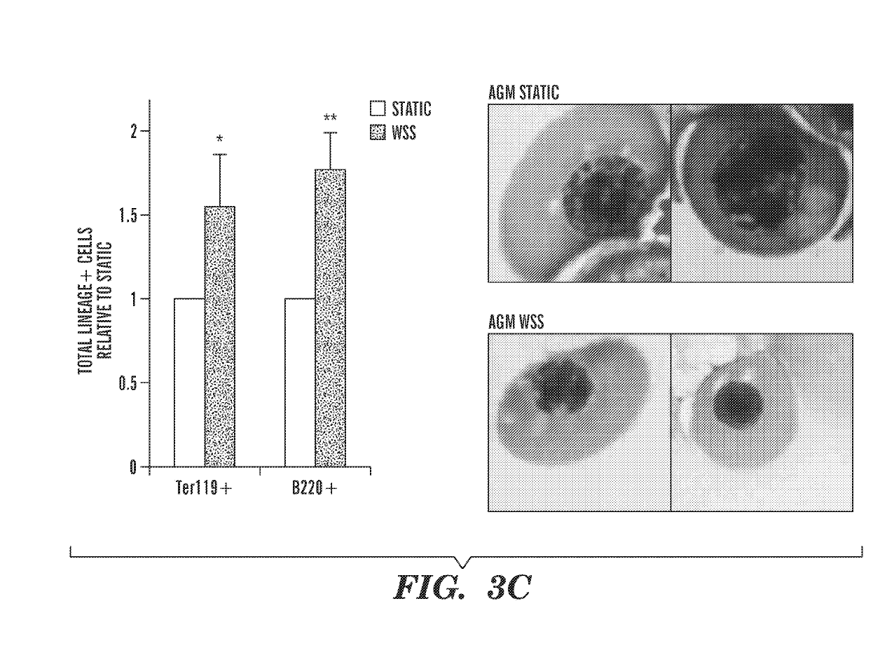

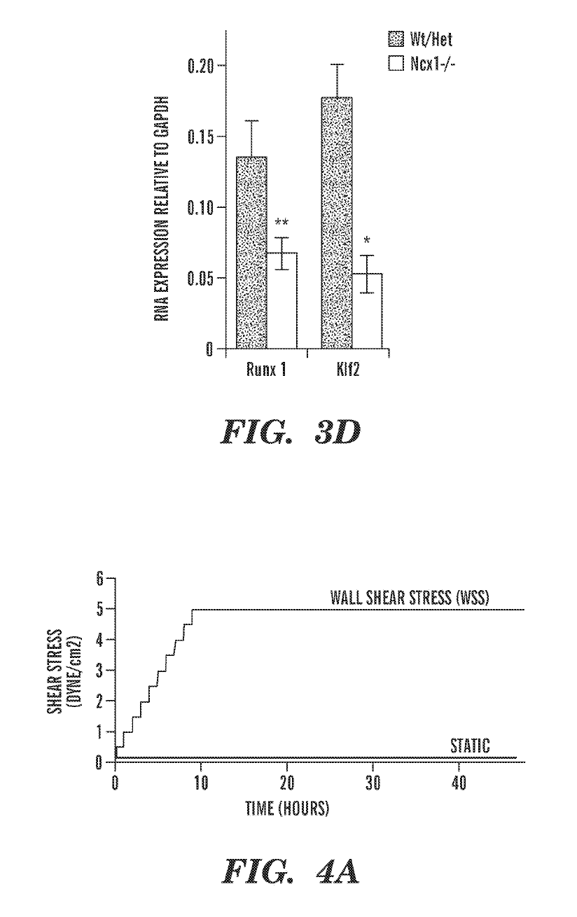

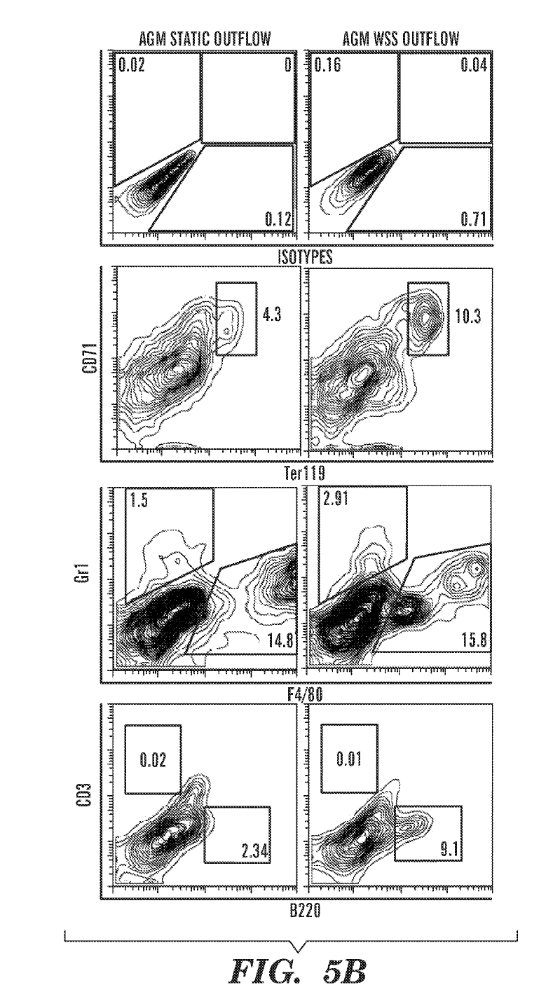

The role of shear stress on the maturation of AGM hematopoietic precursors was also assessed using an optimized 2-dimensional culture system of primary AGM-derived cells. AGM regions from E10.5 murine embryos were disaggregated, plated on gelatinized surfaces, and then exposed to shear stress. As shown in FIG. 3a, shear stress induced an increase in CD31⁺ cells, and upregulated the expression of Runx1 and Klf2, but not of Myb, in FACS-sorted CD41⁺ AGM-derived hematopoietic progenitors. Ferkowicz et al., 130 Devel. 4393-403 (2003). Similar results were observed in the unsorted cell population (FIG. 5a). FACS was used to determine whether or not shear stress impacted specific hematopoietic lineages: there was a shear stress-induced increase in cells positive for the B-lymphocyte marker B220⁺, as well as those positive for the erythroblast marker Ter119 (FIGS. 3c, 5b).

Furthermore, the erythroblasts present on the shear stress-treated cultures displayed morphological features consistent with later stages of erythroid maturation (i.e., pycnotic erythroblasts and a hemoglobinized red pellet versus earlier polychromic erythroblasts present in the static control) (FIG. 3d), indicating accelerated erythroid differentiation in the AGM cultures exposed to shear stress. This is consistent with the strong upregulation of Klf2, a known driver of erythropoiesis. Although there were enhanced erythropoiesis and lymphopoiesis under these conditions, there was no increase in the frequency of CFUs when AGM cultures were exposed to shear stress, likely because progenitors have already sustained biomechanical stimulation in the embryo. Taken together, these observations show that shear stress increases the expression of hematopoietic markers in AGM-derived primary cultures, and modulates embryo-derived hematopoietic differentiation.

That shear stress plays a role in the maturation of embryonic hemogenic sites in vivo was further supported using mouse embryos that fail to initiate circulation due to lack of the Na⁺/Ca²⁺ exchanger, Ncx124. These embryos have been shown to be depleted in hematopoietic CFUs in the AGM, possibly due to a failure of redistribution from the yolk sac (Lux et al., 111 Blood 3435-38 (2007)), and to have a 6-fold reduction in CD41+ hematopoietic progenitors in the placental vasculature. Rhodes et al., 2 Cell Stem Cell 252-63 (2008). These data suggest that the hematopoietic alterations observed in Ncx1^{-/-} embryos may be due, at least in part, to a lack of biomechanical stimulation on vascular hemogenic sites. Thus, whether the master regulator of haematopoiesis, Runx1, is expressed in the AGM region in the absence of circulation, and whether its expression levels are increased by the intravascular fluidic forces generated by the presence of a beating heart, was explored. Importantly, at this developmental stage, Runx1 is exclusively expressed in hemogenic sites within the embryo trunk. Simeone et al., 203 Devel. Dyn. 61-70 (1995); North et al., 126 Devel. 2563-75 (1999). Developing AGMs from Ncx1^{-/-} E9.25 embryos or matched littermate controls were collected, their RNA extracted, and the expression levels of Runx1 and of the shear stress responsive gene Klf2 measured by real-time PCR. As shown in FIG. 3d, both Runx1 and Klf2 were expressed in Ncx1^{-/-} AGMs, but their expression was significantly lower than in littermate controls. Importantly, the expression of the endothelial specific marker, VE-cadherin, was similar in the two groups, suggesting that the content of vascular tissue was similar in both samples. These data strongly suggest that shear stress acts as a determinant of hematopoiesis in the AGM in vivo.

Collectively, these results demonstrate a novel role for biomechanical forces as determinants of hematopoiesis and provide a new framework for understanding how microenviromental cues sustain the specification or maturation of hematopoietic progenitors during embryonic development. Furthermore, they provide new perspectives for the manipulation and production of hematopoietic progenitors in vitro, a key process for the implementation of stem cell-based therapy of hematologic diseases.

The methods of the invention can be used with a variety of animal or human hematopoietic stem cells, including, but not limited to, embryonic stem cells, umbilical cord blood stem cells, unrestricted somatic stem cells derived from human umbilical cord blood, induced stem cells, somatic stem cells, mesenchymal stem cells, mesenchymal progenitor cells, hematopoietic stem cells, hematopoietic lineage progenitor cells, endothelial stem cells, placental fetal stem cells, hemogenic endothelial cells, and endothelial progenitor cells

The hematopoietic stem cell population of the present embodiments may be obtained from iPS cells, embryoid bodies, peripheral blood, cord blood, bone marrow, amniotic fluid, chorionic villus tissue, placenta, or other hematopoietic progenitor cell niches. In particular embodiments, hematopoietic progenitor cells express CD34, CD41, c-kit, Flk1, PECAM, and/or MECA32.

For example, hematopoietic stem cells may be obtained from placenta (U.S. Pat. No. 7,045,148), amniotic fluid (U.S. Pat. No. 7,101,710), or chorionic villus tissues (Bárcena et al., 327 Devel. Bio. 24-33 (2009)). More specifically, an embodiment of the present invention provides for the enhancement of hematopoietic stem cells collected from cord blood or an equivalent neonatal or fetal stem cell source, which may be cryopreserved, for the therapeutic uses of such stem cells upon thawing. See, e.g., WO 2007/112084. Such blood may be collected by several methods known in the art. For example, because umbilical cord blood is a rich source of hematopoietic stem cells (see Nakahata & Ogawa, 70 J. Clin. Invest. 1324-28 (1982); Prindull et al., 67 Acta. Paediatr. Scand. 413-16 (1978); Tchernia et al., 97(3) J. Lab. Clin. Med. 322-31 (1981)), the umbilical cord and placenta provide an excellent source of neonatal blood. The neonatal blood may be obtained by direct drainage from the cord and/or by needle aspiration from the delivered placenta at the root and at distended veins. See, e.g., U.S. Pat. No. 7,160,714; No. 5,114,672; No. 5,004,681; U.S. patent application Ser. No. 10/076,180, U.S. Pub. No. 2003/0032179.

Indeed, umbilical cord blood stem cells have been used to reconstitute hematopoiesis in children with malignant and nonmalignant diseases after treatment with myeloablative doses of chemo-radiotherapy. Sirchia & Rebulla, 84 Haematologica 738-47 (1999). See also Laughlin 27 Bone Marrow Transplant. 1-6 (2001); U.S. Pat. No. 6,852,534. Additionally, it has been reported that stem and progenitor cells in cord blood appear to have a greater proliferative capacity in culture than those in adult bone marrow. Salahuddin et al., 58 Blood 931-38 (1981); Cappellini et al., 57 Brit. J. Haematol. 61-70 (1984).

Alternatively, fetal blood can be taken from the fetal circulation at the placental root with the use of a needle guided by ultrasound (Daffos et al., 153 Am. J. Obstet. Gynecol. 655-60 (1985); Daffos et al., 146 Am. J. Obstet. Gynecol. 985-87 (1983), by placentocentesis (Valenti, 115 Am. J. Obstet. Gynecol. 851-53 (1973); Cao et al., 19 J. Med. Genet. 81-87 (1982)); by fetoscopy (Rodeck, in PRENATAL DIAGNOSIS (Rodeck & Nicolaides, eds., Royal College of Obstetricians & Gynaecologists, London, 1984)). Indeed, the chorionic villus and amniotic fluid, in addition to cord blood and placenta, are sources of pluripotent fetal stem cells (see WO 2003/042405) that may be stimulated by the methods of the present invention.

A hematopoietic stem cell population can be fractionated based, for example, on CD34 or CD41 expression (Mikkola et al., 2003), and enriched for hematopoietic activity by stimulation with shear stress. Regarding cord blood, CD41 antigen is expressed on a fraction of long-term culture initiating cells (LTC-ICs) and long-term reconstituting human stem cells as well as on cells with a B-cells, T-cells, and natural killer (NK)-lymphoid potential derived from cord blood cells. In adults, CD41 expression is more restricted and is essentially present on erythroid and megakaryocyte progenitors, although the CD41⁺ cell population may include some LTC-ICs. Debili et al., 97 Blood 2023-30 (2001). CD41⁺ cells may also be obtained from peripheral blood cells, or expanded from immunoselected CD34⁺ cells from peripheral blood stems cells (PBSCs), by for example, culturing in media with or without serum, supplemented by IL-3, IL-6, IL-11, SCF, TPO, Flt-3 ligand, and MIP-1α. Halle et al., 79 Annals Hematol. 13-19 (2000). Additionally, CD41⁺ cells may be obtained from normal adult human bone marrow, or induced in culture from CD34⁺CD41⁻ bone marrow cells by treatment with thrombopoietin (TPO) or IL-3. Basch et al., 105 Brit. J. Haematol. 1044-54 (1999).

Additionally, a population of stem cells may be harvested, for example, from a bone marrow sample of a subject or from a culture. Harvesting hematopoietic stem cells is defined as the dislodging or separation of cells. This is accomplished using a number of methods, such as enzymatic, non-enzymatic, centrifugal, electrical, or size-based methods, or preferably, by flushing the cells using culture media (e.g., media in which cells are incubated) or buffered solution. The cells are optionally collected, separated, and further expanded generating even larger populations of hematopoietic stem cells and differentiated progeny.

Hematopoietic progenitor cells are optionally obtained from blood products. A blood product includes a product obtained from the body or an organ of the body containing cells of hematopoietic origin. Such sources include unfractionated bone marrow, umbilical cord, peripheral blood, liver, thymus, lymph and spleen. All of the aforementioned crude or unfractionated blood products can be enriched for cells having hematopoietic stem cell characteristics in a number of ways. For example, the more mature, differentiated cells are selected against, via cell surface molecules they express. Optionally, the blood product is fractionated by selecting for CD34⁺ cells. CD34⁺ cells include a subpopulation of cells capable of self-renewal and pluripotentiality. Such selection is accomplished using, for example, commercially available magnetic anti-CD34 beads (Dynal, Lake Success, N.Y.). Unfractionated blood products are optionally obtained directly from a donor or retrieved from cryopreservative storage.

The invention also provides methods of treating a subject in need of improved hematopoietic capability, by stimulating hematopoietic stem cells with shear stress, thereby enhancing proliferation or hematopoietic differentiation of the progenitor cells; and administering the cells to the subject, thereby improving hematopoietic capability.

As used throughout, a subject is an individual. Thus, subjects include, for example, domesticated animals, such as cats and dogs, livestock (e.g., cattle, horses, pigs, sheep, and goats), laboratory animals (e.g., mice, rabbits, rats, and guinea pigs) mammals, non-human mammals, primates, non-human primates, rodents, birds, reptiles, amphibians, fish, and any other animal. The subject is optionally a mammal such as a primate or a human.

The subject referred to herein is, for example, a bone marrow donor or an individual with or at risk for depleted or limited blood cell levels. Optionally, the subject is a bone marrow donor prior to bone marrow harvesting or a bone marrow donor after bone marrow harvesting. The subject is optionally a recipient of a bone marrow transplant. The methods described herein are particularly useful in subjects that have limited bone marrow reserve such as elderly subjects or subjects previously exposed to an immune depleting treatment such as chemotherapy. The subject, optionally, has a decreased blood cell level or is at risk for developing a decreased blood cell level as compared to a control blood cell level.

As used herein the term control blood cell level refers to an average level of blood cells in a subject prior to or in the substantial absence of an event that changes blood cell levels in the subject. An event that changes blood cell levels in a subject includes, for example, anemia, trauma, chemotherapy, bone marrow transplant and radiation therapy. For example, the subject has anemia or blood loss due to, for example, trauma. The subject optionally has depleted bone marrow related to, for example, congenital, genetic or acquired syndrome characterized by bone marrow loss or depleted bone marrow. Thus, the subject is optionally a subject in need of hematopoeisis. Optionally, the subject is a bone marrow donor or is a subject with or at risk for depleted bone marrow.

Hematogenic stem cell manipulation is useful as a supplemental treatment to chemotherapy or radiation therapy. For example, hematogenic progenitor cells are localized into the peripheral blood and then isolated from a subject that will undergo chemotherapy, and after the therapy the cells are returned. Thus, the subject is a subject undergoing or expected to undergo an immune cell-depleting treatment such as chemotherapy, radiation therapy or serving as a donor for a bone marrow transplant. Bone marrow is one of the most prolific tissues in the body and is therefore often the organ that is initially damaged by chemotherapy drugs and radiation. The result is that blood cell production is rapidly destroyed during chemotherapy or radiation treatment, and chemotherapy or radiation must be terminated to allow the hematopoietic system to replenish the blood cell supplies before a patient is re-treated with chemotherapy. Therefore, as described herein, stimulated hematopoietic progenitor cells or blood cells made by the methods described herein are optionally administered to such subjects in need of additional blood cells.

Also provided herein are pharmaceutical compositions comprising one or more hematopoiesis modulators or combinations thereof and a least one pharmaceutically acceptable excipient or carrier. By pharmaceutically acceptable is meant a material that is not biologically or otherwise undesirable, i.e., the material may be administered to a subject or cell, without causing undesirable biological effects or interacting in a deleterious manner with the other components of the pharmaceutical composition in which it is contained. The carrier or excipient is selected to minimize degradation of the active ingredient and to minimize adverse side effects in the subject or cell. The compositions are formulated in any conventional manner for use in the methods described herein. Administration is via any route known to be effective by one of ordinary skill. For example, the compositions is administered orally, parenterally (e.g., intravenously), by intramuscular injection, by intraperitoneal injection, transdermally, extracorporeally, intranasally or topically. The formulation of pharmaceutical compositions is well known in the art. See, e.g., WO 2009/155041; WO 2007/112084; REMINGTON: SCI. & PRACTICE PHARMACY (21st Ed., Troy, ed., Lippincott Williams & Wilkins, Philadelphia, Pa., 2005).

Ex vivo stimulation of hematopoietic progenitor cell population by shear stress adds to our repertoire of compositions and techniques for expanding hematopoietic progenitor cell populations, such that even small amounts of these cells can be expanded enough for transplantation. Consequently, for example, cord blood stem cell transplantation may now be applied to not only children but also adults. Such progenitor cells may be collected from sources including, for example, peripheral blood, cord blood, bone marrow, amniotic fluid, or placental blood.

Alternatively, the hematopoietic stem cell-containing source sample may be harvested and then stored (immediately or after stimulation) in the presence of a hematopoietic progenitor cell modulator, or incubated (before or after stimulation) in the presence of hematopoietic progenitor cell modulator, before cell introduction into a subject. Such storage includes cryopreservation. Further, by increasing the number of hematopoietic cells available for transplantation back into the subject, or to another subject, potentially reduces the time to engraftment, and consequently decreases in the time during which the subject has insufficient neutrophils and platelets, thus preventing infections, bleeding, or other complications.

In vitro expansion of hematopoietic stem cell populations and their differentiating cellular products, according to the present invention, also provides hematopoietic cell sources for drug screening and other research. For instance, currently Plasmodium-infected reticulocytes are obtained from infected individuals, or from precursor cells obtained from peripheral blood mononuclear cells infected in vitro—both limited sources for such cells which are critical for understanding the pathogenesis and treatment of malaria. See, e.g., Chang & Stevenson, 34 Intl. J. Parasitol. 1501-16 (2004); Pichyangkul et al., 172 J. Immunol. 4926-33 (2004). Further regarding anti-malaria drug screening, for example, because artemisinin has poor bioavailability that limits its effectiveness in treating malaria, semi-synthetic and fully synthetic derivatives of artemisinin are being developed. Also, because drug-resistant Plasmodium strains are emerging, it is becoming increasingly important to develop combination therapies. New artemisinin derivatives and combination therapies may be tested in Plasmodium-infected cells that have been produced by the methods of the present invention, and the differentiation and/or expansion of the hematopoietic cell population observed (e.g., erythrocytes).

### EXAMPLES

### Example 1

### Summary

Hemodynamic shear stress in the embryonic aorta was estimated using published fluid dynamic data from the developing mouse embryo and assuming circular Poiseuille flow. See Ji et al., 92 Circ. Res. 133-35 (2003); Phoon et al., 283 Am. J. Physiol. Heart Circ. Physiol. H908-16 (2002); Jones et al, 287 Am. J. Physiol. Heart Circ. Physiol. H1561-69 (2004); Nosek, ESSENTIALS OF HUMAN PHYSIOL.—CARDIAC & CIRCULATORY PHYSIOL. (Gold Standard Multimedia, Inc., Tampa, Fla., 2000). Ainv15 CDX4-inducible mouse embryonic stem cells (iCDX4 ES) were cultured as described (Wang et al., 102 P.N.A.S. 19081-86 (2005)), and differentiated via the hanging drop Embryoid Body method. Differentiated ES cells were plated at 100,000 cm² on 95 cm² plates, and exposed to shear stress in the presence or absence of 2 mM N(G)-nitro-L-arginine methyl ester (L-NAME) or 2 mm N(G)-nitro-D-arginine methyl ester (D-NAME). Pregnant Swiss-Webster mice were purchased from Taconic Farms, Inc. (Germantown, N.Y.). The designation “0.5 days” of gestation was assigned at noon on the day the copulation plug was found. AGM were dissected with a conservative approach, preserving the somites. Two dimensional primary AGM cultures were optimized in the presence of growth factors (ECGS, Heparin, SCF, VEGF, TPO, and Flt3-ligand).

### Example 2

### mESC Culture and Differentiation

Ainv15 CDX4-inducible mouse embryonic stem cells (iCDX4 ES) were cultured on mitomycin-treated Mouse Embryonic Fibroblasts (MEFs) (Chemicon, strain CF-1) and passaged every 2 to 3 days with 0.25% trypsin/EDTA as previously described. Wang et al., 102 P.N.A.S. 19081-86 (2005). To induce differentiation, ES cells were trypsinized, gently dispersed by repetitive pipetting and re-plated for 45 min at 37° C. for MEF depletion. Floating and loosely-adherent cells were gathered, checked for viability with trypan blue, and diluted in Embryoid Body (EB) differentiation media (Iscove's Modified Dulbecco's Medium [IMDM], 10% FCS [STEMCELL Tech., Vancouver, British Columbia, cat #06952], Penicillin 10 units/ml, Streptomycin 10 μg/ml, 1 mM L-Glutamine, Monothiolglycerol 3.8 nl/ml, Fe-saturated transferrin 0.2 mg/ml, Ascorbic Acid 0.5 mg/ml), to a final concentration of 6.7×10³ cell/ml. EBs were cultured via the hanging drop method in 15 μl drops on 15 cm bacterial dishes. Typically, 100 plates of EBs were used per experiment. After 48 hr, EBs were pooled into calcium and magnesium containing warm PBS and collected by precipitation in 50 ml Falcon tubes. Pooled EBs were transferred to 10 cm tissue culture dishes containing 10 ml of EB differentiation media each, placed on an orbital shaker moving at 75 rev/min, and incubated for an additional 30 hr. Following this second culture phase, day 3.25-EBs were collected by precipitation, washed two times in 30 ml calcium-free PBS and trypsinized for 1 min in a 1:1 dilution of calcium-free PBS and 0.25% Trypsin-EDTA. Trypsinization was blocked by addition of 10 ml EB differentiation media.

### Example 3

### Hemodynamic Shear Stress Estimation

Wall shear stress acting along the dorsal aorta transiently increases during embryonic development once the heart begins beating. This increase in shear stress may be attributed to both an increase in blood flow velocity, a result of increasing cardiac output (Ji et al., 92 Circ. Res. 133-35 (2003); Phoon et al., 283 Am. J. Physiol. Heart Circ. Physiol. H908-16 (2002)), and an increase in viscosity, an effect of increasing hematocrit. Jones et al., 287 Am. J. Physiol. Heart Circ. Physiol. H1561-9 (2004). During the first two embryonic days following the initiation of a beating heart (E8-E10), the hematocrit rises, but remains below 20%. This change has an insignificant contribution to blood viscosity (Nosek, 2000), and therefore, at these early time points blood viscosity can be assumed constant and equal to about 0.015 dyne sec/cm².

Ultrasound biomicroscopy data (Phoon et al., 2002), has shown that blood flow in the developing embryo dorsal aorta is laminar throughout the cardiac cycle with minimal skewing of the spatial velocity profile, as determined by calculation of non-dimensional fluid dynamic parameters at E11.5, including the Reynolds number (157), Womersley parameter (0.63) and Dean number (39), respectively. Assuming such fluid dynamic parameters remain valid for earlier time-points during development, as well as that the instantaneous blood velocity profile is parabolic, the dorsal aorta wall shear stress was estimated based on circular Poiseuille flow in which the wall shear stress, τ, is calculated by τ=8 μV/D, where μ is the apparent viscosity (dyne×sec/cm²), V (mm/sec) is the time-average centerline blood flow velocity, and D (mm) is the vessel diameter. A similar strategy has been used to estimate embryonic shear stress levels in the developing embryo (E8.5-10.5). Jones et al., 2004.

### Example 4

### Mouse Embryonic Stem Cell-Exposure to Shear Stress

Differentiated ES cells were plated at 100,000 cm² on horizontal 95 cm² plates, compatible with a Dynamic Flow System. Blackman et al., 124 J. Biomech. Engin. 397-407 (2002). The Dynamic Flow System is a modified cone and cell plate device with a computer-controlled user interface, in which a cone is controlled by a variable-speed precision motor in a single well of a multi-well plate, as described in PCT/US2010/021002 and U.S. patent application Ser. No. 12/687,717, each filed Jan. 14, 2010 and incorporated herein.

The system includes an array of mechanical tips that each correspond to one of the wells in a multi-well plate, an interface for positioning each tip within its corresponding well, and a separate driver associated with each tip for driving the tip to impart a shear stress pattern in its corresponding well. Each of the wells is at least partially filled with a liquid (e.g., a cell culture medium) and contains biological cells. A separate mechanical tip is disposed in each of the wells. Each mechanical tip is then separately rotated to transmit a shear force through the liquid to the cells. In addition, each mechanical tip (whose bottom surface may optionally have a flat center and an overall conical shape) may be positioned so as not to contact a surface of its corresponding well and/or so as not to contact any of the cells grown therein.

The shear stress pattern delivered to the biological cells adhered to the well bottom is dependent on the distance, h, between the rotating cone of the tip and the bottom of the wells of the well plate. Given the dimensions of the device and the hemodynamic settings in which the device may be used, a 1 μm error in h will produce a 1% error in the shear stress applied. A post of precise length may be incorporated to the cone and will maintain a constant distance, h. The entire tip assembly will be spring loaded such that the post remains in contact with the well bottom. Bearings internal to the tip will allow the post to remain stationary as the cone rotates around it. This self adjusting configuration maintains a constant distance between cone and well bottom despite variations in commercially available well plates (e.g., Nunc brand plates, Thermo Fisher Sci., Inc., Rochester, N.Y.).

Hemodynamic shear stress waveforms are applied within all the wells in a well plate by arraying one motor, tip, and collar for each well. A gasket between the motor plate and collar ensures that all collars are seated in their individual wells. Other methods for applying shear in an array are possible. For example, fewer motors may be used with mechanical linkages, such as pulleys or chains, so that each motor drives multiple cones in parallel. The cones may alternatively be driven by non-mechanical methods, including electromagnetic or pneumatic actuation. The flow system can be adapted to accommodate a wide variety of commercially available well plates, custom-built well plates, and well plates with different dimensions and numbers of wells.

At least one of the drivers associated with a mechanical tip is a variable-speed precision motor, such as a stepper motor, for example, an Arsape AM-0820 high-precision micro-stepper motor (MicroMo Electronics, Inc., Clearwater, Fla.). Electronics for the motor include the circuitry, hardware, and software for driving the motors with specific velocity profiles, for example microstepping drivers (Allegro MicroSystems, Inc., Worcester, Mass.), microcontrollers (Microchip Tech., Inc., Chandler, Ariz.) and an external power supply (e.g., a VPM-S300 12V DC power supply, V-Infinity, LLC, Tualatin, Oreg.).

The Flow System may further include means for regulating a level of CO₂ and/or O₂ in the environment surrounding the well array (for example, a mixed air pressurized tank and flow regulator available from, e.g., Airgas, Inc., Radnor, Pa.), and means for controlling the temperature and humidity in that environment. For example, the well array may be positioned within a temperature-controlled fluid bath, and a heater may be employed to control the temperature of the fluid bath (for example, a heater such as a 5×6 inch 2.5 Watt/in² flexible silicone fiberglass insulated heater with a temperature measurement thermocouple, and a heater input power adjustment with a ramp/soak temperature controller, all from Omega Engin., Inc., Stamford, Conn.). Typically, the temperature of the fluid bath is maintained at approximately 37° C. The well array may include, for example, 96 wells defined within a well plate, e.g., commercially available 96-well plates.

The shear stress patterns may mimic physiological hemodynamic waveforms present in the circulatory system of an organism. The design parameters (e.g., cone angle, plate diameter, and medium viscosity) had been evaluated to ensure that the flow is laminar, and that the shear stress is directly proportional to the angular velocity of the cone. Therefore, precise control of the cone rotation allows the device to simulate various shear stress waveforms. See also Blackman et al., 2002.

A general strategy for applying hemodynamic waveforms to cultured cells may involve the following steps: First, each well of the array of wells may be at least partially filled with a cell culture medium and cells may then be grown therein. Then, the cone of each motorized tip unit may be precisely located in each well to a predetermined height above the cell surface. Then, each cone may be separately driven (e.g., rotated) by its respective motor, thereby transmitting a shear force through the cell culture medium to the cells. This device can impart shear stress patterns having magnitudes ranging from 0 dyne/cm² to 100 dyne/cm², inclusive. For example, shear stress pattern magnitudes may range from 0 dyne/cm² to 5 dyne/cm², 0 dyne/cm² to 10 dyne/cm², 0 dyne/cm² to 15 dyne/cm², 0 dyne/cm² to 20 dyne/cm², 0 dyne/cm² to 25 dyne/cm², 0 dyne/cm² to 30 dyne/cm², 0 dyne/cm² to 35 dyne/cm², 25 dyne/cm² to 45 dyne/cm², inclusive, or the magnitude applied to the cell about 35 dyne/cm². The shear stress may be applied for periods of time ranging from 1 hr to 72 hr, inclusive, such as from 1 hr to 12 hr, 1 hr to 36 hr, 1 hr to 48 hr, inclusive. Magnitudes and times may also be varied within these parameters: For example, cells may be stimulated at an initial 0 dyne/cm² which is gradually increased to about 7 dyne/cm² over a period of about 12 hr, then stimulated at about 7 dyne/cm² shear stress, constantly, for about 36 hr; cells may be stimulated at an initial 0 dyne/cm² which is gradually increased to about 5 dyne/cm² over a period of 12 hr, then stimulated at about 5 dyne/cm² shear stress, constantly, for an additional 36 hr; cells may be stimulated at an initial 0 dyne/cm² which is gradually increased to about 3 dyne/cm² over a period of 12 hr, then stimulated at about 3 dyne/cm² shear stress, constantly, for an additional 36 hr.

A large-scale (approximately 150-mm diameter) cone-and-plate shear machine has been used to apply hemodynamic waveforms to cultured human endothelial cells in the context of several specific experimental goals. The cone in the large-scale machine is conical. With this geometry, the shear is constant over the surface of a dish, and the magnitude τ of this shear may calculated according to the equation:

$$τ = \frac{μΩ}{tan θ}$$

where μ is the viscosity of the fluid in the dish, Ω is the rotational speed of the cone, and θ is the angle between the cone and the dish. Both the large and single-well scale devices can provide a constant shear of approximately 35 dyne/cm² delivered over the majority of the well surface with a cone angle θ of approximately 10° and a rotational speed Ω for the cone of approximately 35 rotations/sec.

More specifically, in the instant approach, plates were coated with a thick layer of 1% gelatin (DIFCO #214340, Becton Dickinson). After 12 hr, cells were washed three times with 10 ml of warm PBS with calcium and incubated for 45 min with EB differentiation media with or without 2 mM N(G)-nitro-L-arginine methyl ester (L-NAME) or 2 mM N(G)-nitro-D-arginine methyl ester (D-NAME) (Sigma-Aldrich). Cells to be moved to the dynamic flow system were incubated in 7 ml of media, and static controls incubated in 20 ml of media. Following 45 min incubation, cells were either moved to the Dynamic Flow System and exposed to shear stress or maintained in a standard cell culture incubator. The shear stress ramped up in a step-wise fashion from 0 dyne/cm² to 5 dyne/cm² over a period of 12 hr, in order to minimize cell loss during the transition from static to dynamic culture. Following the 12 hr ramping period, the cells were then exposed to a constant 5 dyne/cm² shear stress for 36 hr. In cells exposed to shear stress, culture media with or without D-NAME or L-NAME were automatically exchanged and stored at 37° C. and 5% CO₂. In static controls, culture medium was replaced after 24 hr and stored in 75 cm² flasks in a cell culture incubator. After 48 hr exposure to shear stress or growth under static conditions, cells were detached by 10 min incubation with a 1:1 dilution of Trypsin-Versene mixture (Cambrex, #17-161E) and Hank's Balanced Salt Solution.

### Example 5

### RNA Extraction and Quantitative Real-Time PCR

Total RNA was extracted with a RNEASY® Micro Kit (QIAGEN, Inc., Valencia, Calif.), according to manufacturer's instructions. For RNA extraction from CD41⁺/cKIT⁺ cells, approximately 7,000 cells were sorted directly into 300 μl of Lysis Buffer. Reverse transcription of RNA was performed using MULTISCRIBE™ DNA polymerase (Applied Biosystems, Foster City, Calif.), according to manufacturer's instructions. When processing FACS-sorted cells, RNA was eluted in 20 μl and all collected RNA was used in 50 μl reverse transcription reaction. Real Time TAQMAN® PCR (Applied Biosystems) was performed in 20 μl reactions with primers provided by Applied Biosystems, according to manufacturer's instructions.

### Example 6

### FACS Sorting

Cells were incubated with Fc Block antibody (BD 553141, dilution 1:100) and then stained with anti CD41 antibody (BD 553848, 1:150 dilution), anti cKIT antibody (BD 553356, 1:150 dilution) and 7AAD (BD 559925, 1:75 dilution) and sorted with a FACSARIA™ cell analyzer system (BD Biosciences, San Jose, Calif.).

### Example 7

### Methylcellulose Colony Forming Unit Assay

Methylcellulose assay was performed as previously described and scored on days 8 to 10 post-plating. Wang et al., 102 P.N.A.S. 19081-86 (2005).

### Example 8

### Animal Treatments

Day-7 post-coitum timed-pregnant Swiss-Webster mice were purchased from Taconic Farms. Noon on the day the copulation plug was found was designated 0.5 days. On day E8.5 (day 9 post coitum) mice were intraperitoneally injected with 300 mg/kg of L-NAME or D-NAME (SIGMA) dissolved in sterile, apyrogenic saline solution using a dose volume of 10 ml/kg. The injection was repeated after 24 hr. Exactly 24 hr after the second injection, mice were euthanized with CO₂, embryos were harvested, and a conservative dissection of the AGM was performed. Embryo trunks were dissociated with 0.1% dispase and analyzed with the methylcellulose assay. Experiments were carried out with Institutional Animal Care and Use Committee approval from Harvard Medical School or Children's Hospital, Boston.

### Example 9

### Primary AGM Culture

E10.5 murine embryos were collected in PBS, a conservative dissection of the AGM region preserving the somites was performed, and embryo derived tissues disaggregated by incubation for 1 hr in 0.1% dispase (Gibco) at 37° C. Typically, thirty embryos were used per condition to enable the coverage of the full Dynamic Flow System cell growth surface area (95 cm²) at a density of 100,000 cells/cm². AGM-derived cells were plated on gelatinized surfaces in IMDM (10% FCS, 1×NEAA (Gibco), 1×β-mercaptoethanol (Gibco), 1 mM L-Glutamine, Penicillin 10 units/ml, Streptomycin 10 μg/ml, ECGS 50 μg/ml, Heparin 100 μg/ml, SCF 100 ng/mL, VEGF 40 ng/mL, TPO 40 ng/mL and Flt3-ligand 100 ng/mL) overnight. Media supplementation was then reduced to ECGS 25 μg/ml, Heparin 50 μg/ml, SCF 25 ng/ml, VEGF 20 ng/ml, TPO 10 ng/ml and Flt3-ligand 25 ng/ml. Cells were exposed to shear stress and harvested as described herein for mES-derived cells. Non-adherent cells were gathered by centrifugation.

All the statistical analyses were performed utilizing un-paired two-tailed Student's t-test assuming experimental samples of equal variance.

### Example 10

### Hematopoietic Modulators Combine with Biomechanical Simulation

As described elsewhere (see, e.g., WO 2009/155041; WO 2007/112084), modulators of hematopoiesis may be used to enhance differentiation and/or expansion of hematopoietic progenitor cells. Hence, in some embodiments, such modulators are used in combination with the methods of the present invention. In other words, such modulators may be used before, during, or after biomechanical (e.g., shear stress) stimulation of hematopoietic progenitor cells to further enhance differentiation and/or expansion of stimulated cells.

Example compounds that can be used in combination with biomechanical stimulation to promote hematopoiesis include, but are not limited to, Atenolol, Digoxin, Doxazosin, Doxycycline, Fendiline, Hydralazine, β-hydroxyoctadecadienoic acid (13(s)-HODE), Lanatoside C, NG-monomethyl-L-arginine (L-NMMA), Metoprolol, Nerifolin, Nicardipine, Nifedipine, Nitric oxide (NO) or NO signaling pathway agonists, 1H-[1,2,4]oxadiazolo-[4,3-a]quinoxalin-1-one (ODQ), Peruvoside, Pindolol, Pronethalol, Synaptosomal protein (SNAP), Sodium Nitroprusside, Strophanthidin, Todralazine, 1,5-Pentamethylenetetrazole, Prostaglandin E 2 (PGE2), PGE2 methyl ester, PGE2 serinol amide, 11-deoxy-16,16-dimethyl PGE2, 15(R)-15-methyl PGE2, 15(S)-15-methyl PGE2, 6,16-dimethyl PGE2, 16,16-dimethyl PGE2 p-(p-acetamidobenzamido) phenyl ester, 16-phenyl tetranor PGE2, 19(R)-hydroxy PGE2, Prostaglandin B2, Prostacyclin (PGI2, epoprostenol), 4-Aminopyridine, 8-bromo-cAMP, 9-deoxy-9-methylene PGE2,9-deoxy-9-methylene-16,16-dimethyl PGE2, a PGE2 receptor agonist, Bapta-AM, Benfotiamine, Bicuclline, (2′Z,3′E)-6-Bromoindirubin-3′-oxime (BIO), Bradykinin, Butaprost, Cay10397, Chlorotrianisene, Chlorpropamide, Diazoxide, Eicosatrienoic Acid, Epoxyeicosatrienoic Acid, Flurandrenolide, Forskolin, Gaboxadol, Gallamine, Indanyloxyacetic acid 94 (IAA 94), Imipramine, Kynurenic Acid, L-Arginine, Linoleic Acid, LY171883, Mead Acid, Mebeverine, 12 Methoxydodecenoic acid, N-Formyl-Met-Leu-Phe, Prostaglandin E2 receptor EP2-selective agonist (ONO-AE1-259), Peruvoside, Pimozide, Pindolol, Sodium Nitroprusside, Sodium Vanadate, Strophanthidin, Sulprostone, Thiabendazole, Vesamicol, 1,2-Didecanoyl-glycerol (10:0), 11,12 Epoxyeicosatrienoic acid, 1-Hexadecyl-2-arachidonoyl-glycerol, 5-Hydroxydecanoate, 6-Formylindolo[3,2-B]carbazole, Anandamide (20:3,n-6), Carbacyclin, Carbamyl-Platelet-activating factor (C-PAF), S-Farnesyl-L-cysteine methyl ester, or derivatives of any of these.

We claim:

1. A method for promoting growth, differentiation and/or maturation of hematopoietic progenitors or hematopoietic cells comprising exposing a population of cultured cells to at least one external biomechanical stimulus.

2. The method of claim 1, wherein the at least one stimulus is wall shear stress ranging from 0 dyne/cm² to 150 dyne/cm², inclusive.

3. The method of claim 2, wherein the stimulus ranges from 0 dyne/cm² to 100 dyne/cm², from 0 dyne/cm² to 35 dyne/cm², from 0 dyne/cm² to 10 dyne/cm², or from 0 dyne/cm² to 5 dyne/cm², inclusive.

4. The method of claim 2, wherein the cells are be exposed for at least 1 hour to 72 hours, inclusive.

5. The method of claim 4, wherein the cells are exposed for about 48 hours.

6. The method of claim 1, wherein the at least one stimulus is circumferential stress.

7. The method of claim 1, wherein the at least one stimulus is hydrodynamic pressure.

8. The method of claim 1, wherein the cells exposed to biomechanical stimulation are derived from differentiation of embryonic stem cells, differentiation of induced pluripotent stem cells, differentiation of somatic stem cells, the vascular wall, peripheral blood, cord blood, bone marrow, amniotic fluid, chorionic villa, placenta, hemogenic endothelium, or from other cell populations endowed with spontaneous or inducible hematopoietic potential.

9. The method of claim 8, wherein the mechanical stimulation is shear stress ranging from 0 dyne/cm² to 100 dyne/cm².

10. The method of claim 9, wherein shear stress is applied in addition to at least one chemical, physical, or biological stimulus.

11. The method of claim 1, wherein the cultured cells are exposed to at least one external biomechanical stimulus for the purpose of inducing hematopoietic expansion or differentiation or maturation through increases in the expression of Runx1/Cbfa2, Myb/Cbfb, and/or Kfl2.

12. The method of claim 2, wherein the cultured cells are exposed to wall shear stress for up to 48 hours.

13. The method of claim 1 further comprising the step of contacting the cultured cells with a chemical modulator of hematopoiesis.

14. A method for producing, hematopoietic cells in a subject, comprising obtaining a cell population enriched in hematopoietic precursors; exposing the cell population enriched in hematopoietic precursors to biomechanical stress to stimulate hematopoietic maturation, expansion and/or differentiation; and administering the stimulated population to the subject.

15. (canceled)

16. (canceled)

17. A method for producing cells of the hematopoietic lineage in a subject, comprising obtaining stem cells; differentiating the cells; exposing the cells derived from this differentiation to shear stress to stimulate hematopoietic maturation, expansion or differentiation of the cells; and administering the stimulated cell population to the subject.

18. The method of claim 17, wherein the stem cells are induced pluripotent stem cells, autologous induced pluripotent stem cells, autologous multipotent stem cells, or allogeneic multipotent stem cells.

19. The method of claim 14, wherein the subject is a human.

20. The method of claim 14, further comprising contacting the cells with a chemical modulator of hematopoiesis.

21. (canceled)
